(19) United States
(12) Patent Application Publication
(10) Pub. No.: US 2017/0347654 A1
(43) Pub. Date: Dec. 7, 2017

(54) Herbicidal Compounds

(71) Applicant: SYNGENTA PARTICIPATIONS AG, Basel (CH)

(72) Inventors: Stephen Edward SHANAHAN, Bracknell, Berkshire (GB); Jeffrey Steven WAILES, Bracknell Berkshire (GB); Jonathan Wesley Paul DALLIMORE, Bracknell Berkshire (GB); James Nicholas SCUTT, Bracknell, Berkshire (GB)

(73) Assignee: Syngenta Participations AG, Basel (CH)

(21) Appl. No.: 15/538,737

(22) PCT Filed: Dec. 15, 2015
(86) PCT No.: PCT/EP2015/079879

§ 371 Date: Jun. 22, 2017

(30) Foreign Application Priority Data
Dec. 22, 2014 (GB) 1422895.1

(51) Int. Cl. A01N 43/90; C07D 513/04

(52) U.S. Cl. CPC A01N 43/90; C07D 513/04

(57) ABSTRACT

The present invention relates to herbicidally active thiazolopyridinones, in particular benzyloxy-substituted-phenyl-thiazolopyridinone derivatives, of formula (I), wherein R¹, R², G, X, Y, Z and n are as defined herein. The invention further provided processes for their preparation, and compositions comprising such derivatives. The invention also extends to the use of such compounds and compositions as herbicides, i.e. for the control of undesirable plant growth. In particular such compounds and compositions may be used in controlling weeds, such as broad-leaved dicotyledonous weeds, in crops of useful plants.

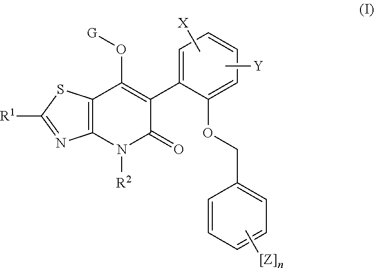

The present invention relates to herbicidally active thiazolopyridinones, in particular benzyloxy-substituted-phenyl-thiazolopyridinone derivatives, processes for their preparation, and compositions comprising such derivatives. The invention also extends to the use of such compounds and compositions as herbicides, i.e. for the control of undesirable plant growth. In particular such compounds and compositions may be used in controlling weeds, such as broad-leaved dicotyledonous weeds, in crops of useful plants.

The prior art describes various herbicidally active pyridinones forming a condensed ring system with either five- or six-membered heterocyclic rings. For example WO2011/051212 discloses pyridinones which are condensed with selected five-membered heterocycles and which are substituted in the 3 position of the pyridine ring by aryl and heteroaryl radicals. WO 2012/028582 discloses pyridinones which are condensed with selected five- and six-membered heterocycles and which are substituted in the 3 position of the pyridine ring by aryl radicals. WO2013/144096 describes herbicidally and insecticidally active thiazolopyridinones, which are substituted in the 3 position of the pyridine ring by aryl or heteroaryl radicals.

The present invention is based on the finding that thiazolopyridinones of general formula (I) as shown below, wherein the thiazolopyridinone moiety is substituted in the 3 position of the pyridine ring with a benzyl-oxy-substituted aryl moiety, are particularly efficacious, and selective, herbicides.

Thus, in a first aspect there is provided a compound of formula (I)

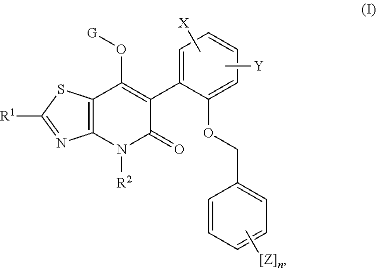

or a salt or N-oxide thereof, wherein: R¹ is hydrogen, halogen, nitro, cyano, or selected from the group consisting of : C₁-C₆alkyl, C₂-C₆ alkenyl, C₁-C₆ alkoxy C₁-C₆ alkylthio, C₁-C₆alkylsulfinyl, C₁-C₆alkylsulfonyl, C₃-C₆cycloalkyl, C₁-C₆ alkoxy-C₁-C₆alkyl-, C₁-C₆alkoxy-C₁-C₆alkoxy-, di-C₁-C₆alkoxy-C₁-C₆alkyl, and C₁-Cₛalkylthio-C₁-C₆alkyl, each of which is optionally substituted by 1-3 halogen atoms;
R² is hydrogen; or C₁-C₆alkyl, C₂-C₆ alkenyl, C₃-C₆alkynyl C₃-C₆cycloalkyl, C₁-C₆ alkoxy-C₁-C₆alkyl-, C₁-C₆alkoxy-C₁-C₆alkoxy-, di-C₁-C₆alkoxy-C₁-C₆alkyl, C₁-C₆alkylthio-C₁-C₆alkyl, C₁-C₆alkylsulfinyl-C₁-C₆alkyl, C₁-C₆alkylsulfonyl-C₁-C₆alkyl, C₃-C₆cycloalkyl-C₁-C₆alkyl, or cyano-C₁-C₆alkyl, each of which is optionally substituted by 1-3 halogen atoms; or phenyl optionally substituted by 1-3 substituents independently selected from halogen, C₁-C₆ alkyl and C₁-C₆ alkoxy; or benzyl optionally substituted by 1-3 substituents independently selected from halogen, C₁-C₆ alkyl and C₁-C₆ alkoxy; G is hydrogen, C₁-C₁₀alkyl, C₂-C₁₀alkenyl, C₂-C₁₀alkynyl or aryl-C₁-C₄-alkyl- or aryl-C₁-C₄alkyl- wherein the aryl moiety is substituted by one to five substituents independently selected from halo, cyano, nitro, C₁-C₆alkyl, C₁-C₆haloalkyl or C₁-C₆alkoxy, or C(O)R³;
R³ is C₁-C₁₀alkyl, C₃-C₁₀cycloalkyl, C₃-C₁₀cycloalkyl-C₁-C₁₀alkyl-, C₁-C₁₀haloalkyl, C₂-C₁₀alkenyl, C₂-C₁₀alkynyl, C₁-C₄alkoxy-C₁-C₁₀alkyl-, C₁-C₁₀alkylthio-C₁-C₄alkyl-, C₁-C₁₀alkoxy, C₂-C₁₀alkenyloxy, C₂-C₁₀alkynyloxy, C₁-C₁₀alkylthio-, NR⁵R⁶, N—C₁-C₄alkyl-amino-, N,N-di-(C₁-C₄alkyl)-amino-, aryl or aryl substituted by 1-3 R⁴, which may be the same or different, heteroaryl or heteroaryl substituted by 1-3 R⁴, which may be the same or different, aryl-C₁-C₄alkyl- or aryl-C₁-C₄alkyl- wherein the aryl moiety is substituted by 1-3 to three R⁴, which may be the same or different, heteroaryl-C₁-C₄alkyl- or heteroaryl-C₁-C₄alkyl- wherein the heteroaryl moiety is substituted by 1-3 R⁴, which may be the same or different, aryloxy- or aryloxy-substituted by 1-3 R⁴, which may be the same or different, heteroaryloxy- or heteroaryloxy-substituted by 1-3 R⁴, which may be the same or different, arylthio- or arylthio-substituted by 1-3 R⁴, which may be the same or different, or heteroarylthio- or heteroarylthio-substituted by one to three R⁴, which may be the same or different;
each R⁴ is independently halo, cyano, nitro, C₁-C₁₀alkyl, C₁-C₄haloalkyl, C₁-C₁₀alkoxy, C₁-C₄alkoxycarbonyl-, C₁-C₄haloalkoxy, C₁-C₁₀alkylthio-, C₁-C₄haloalkylthio-, C₁-C₁₀alkylsulfinyl-, C₁-C₄haloalkylsulfinyl-, C₁-C₁₀alkylsulfonyl-, C₁-C₄haloalkylsulfonyl-, aryl or aryl substituted by 1-5 substituents independently selected from halo, cyano, nitro, C₁-C₆alkyl, C₁-C₆haloalkyl or C₁-C₆alkoxy, or heteroaryl or heteroaryl substituted by 1-4 substituents independently selected from halo, cyano, nitro, C₁-C₆alkyl, C₁-C₆haloalkyl or C₁-C₆alkoxy;
R⁵ and R⁶ are independently selected from the group consisting of C₁-C₆ alkyl and C₁-C₆ alkoxy, or R⁵ and R⁶ together can form a morpolinyl ring;
X and Y are each independently hydrogen, C₁-C₃ alkyl, C₁-C₃ alkoxy, C₁-C₃haloalkyl, C₁-C₃haloalkoxy, or halogen;
Z is C₁-C₃ alkyl, C₁-C₃ alkoxy, C₁-C₃haloalkyl, C₁-C₃haloalkoxy, or halogen; and n is an integer of 0, 1, 2, 3, 4, or 5.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

Similarly, where there are di-substituted alkenes, these may be present in E or Z form or as mixtures of both in any proportion.

Furthermore, compounds of formula (I) may be in equilibrium with alternative tautomeric forms. For example, a compound of formula (I-i), i.e. a compound of formula (I) wherein R² is hydrogen, and G is hydrogen, can be drawn in at least five tautomeric forms:

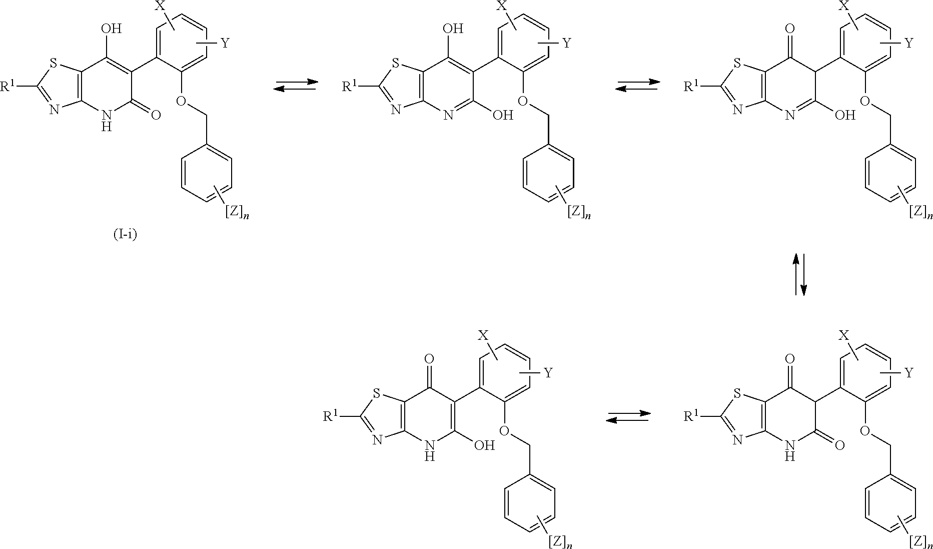

It should be appreciated that all tautomeric forms (single tautomer or mixtures thereof), racemic mixtures and single isomers are included within the scope of the present invention.

Each alkyl moiety either alone or as part of a larger group (such as alkoxy, alkylthio, alkoxycarbonyl, alkylcarbonyl, alkylaminocarbonyl, or dialkylaminocarbonyl, et al.) may be straight-chained or branched. Typically, the alkyl is, for example, methyl, ethyl, n-propyl, isopropyl, n-butyl, sec-butyl, isobutyl, tert-butyl, n-pentyl, neopentyl, or n-hexyl. The alkyl groups are generally C₁-C₆alkyl groups (except where already defined more narrowly), but are preferably C₁-C₄alkyl or C₁-C₃alkyl groups (except where already defined more narrowly), and, more preferably, are C₁-C₂alkyl groups (such as methyl).

Alkenyl and alkynyl moieties can be in the form of straight or branched chains, and the alkenyl moieties, where appropriate, can be of either the (E)- or (Z)-configuration. The alkenyl or alkynyl are typically C₂-C₄alkenyl or C₂-C₄alkynyl, more specifically vinyl, allyl, ethynyl, propargyl or prop-1-ynyl. Alkenyl and alkynyl moieties can contain one or more double and/or triple bonds in any combination; but preferably contain only one double bond (for alkenyl) or only one triple bond (for alkynyl).

Preferably, the term cycloalkyl refers to cyclopropyl, cyclobutyl, cyclopentyl or cyclohexyl.

In the context of the present specification the term “aryl” preferably means phenyl. The term “heteroaryl” as used herein means an aromatic ring system containing at least one ring heteroatom and consists of a single ring. Preferably, single rings will contain 1, 2 or 3 ring heteroatoms selected independently from nitrogen, oxygen and sulfur. Typically “heteroaryl” is furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, or 1,3,5-triazinyl.

Heterocyclyl groups and heterocyclic rings (either alone or as part of a larger group, such as heterocyclyl-alkyl-) are ring systems containing at least one heteroatom and can be in mono- or bi-cyclic form. Preferably, heterocyclyl groups will contain up to two heteroatoms which will preferably be chosen from nitrogen, oxygen and sulfur. Examples of heterocyclic groups include oxetanyl, thietanyl, azetidinyl and 7-oxa-bicyclo[2.2.1]hept-2-yl. Heterocyclyl groups containing a single oxygen atom as heteroatom are most preferred. The heterocyclyl groups are preferably 3- to 8-membered, more preferably 3- to 6-membered rings.

Halogen (or halo) encompasses fluorine, chlorine, bromine or iodine. The same correspondingly applies to halogen in the context of other definitions, such as haloalkyl or halophenyl.

Haloalkyl groups having a chain length of from 1 to 6 carbon atoms are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl, heptafluoro-n-propyl and perfluoro-n-hexyl.

Alkoxy groups preferably have a chain length of from 1 to 6 carbon atoms. Alkoxy is, for example, methoxy, ethoxy, propoxy, isopropoxy, n-butoxy, isobutoxy, sec-butoxy or tert-butoxy or a pentyloxy or hexyloxy isomer, preferably methoxy and ethoxy. It should also be appreciated that two alkoxy substituents present on the same carbon atom.

Haloalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₁-C₆-alkylthio (C₁-C₆alkyl-S—) is, for example, methylthio, ethylthio, propylthio, isopropylthio, n-butylthio, isobutylthio, sec-butylthio or tert-butylthio, preferably methylthio or ethylthio.

C₁-C₆-alkylsulfinyl (C₁-C₆alkyl-S(O)—) is, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₆-alkylsulfonyl (C₁-C₆alkyl-S(O)₂—) is, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

The present invention also includes agronomically acceptable salts that the compounds of formula (I) may form with amines (for example ammonia, dimethylamine and triethylamine), alkali metal and alkaline earth metal bases or quaternary ammonium bases. Among the alkali metal and alkaline earth metal hydroxides, oxides, alkoxides and hydrogen carbonates and carbonates used as salt formers, emphasis is to be given to the hydroxides, alkoxides, oxides and carbonates of lithium, sodium, potassium, magnesium and calcium, but especially those of sodium, magnesium and calcium. The corresponding trimethylsulfonium salt may also be used. The compounds of formula (I) according to the invention also include hydrates which may be formed during the salt formation.

Preferred values of R¹, R², G, R³, R⁴, R⁵, R⁶, R⁷, X, Y, Z, and n are as set out below, and a compound of formula (I) according to the invention may comprise any combination of said values. The skilled man will appreciate that values for any specified set of embodiments may combined with values for any other set of embodiments where such combinations are not mutually exclusive.

Preferably R¹ is hydrogen, or selected from the group consisting of: C₁-C₆-alkyl, C₁-C₆-alkoxy, C₁-C₆alkoxy-C₁-C₆alkoxy-, C₁-C₆-alkylthio, C₁-C₆-alkylsulfonyl, C₃-C₆-cycloalkyl, C₁-C₆-alkoxy-C₁-C₆-alkyl and C₁-C₆-alkylthio-C₁-C₆-alkyl, each of which is optionally substituted by 1-3 halogen atoms. More preferably R¹ is selected from hydrogen C₁-C₆-alkyl, C₁-C₃-alkoxy, C₁-C₃alkoxy-C₁-C₃alkoxy-, C₁-C₃-alkylthio, C₁-C₃-alkylsulfonyl, C₃-C₄-cycloalkyl, C₁-C₃alkoxy-C₁-C₃-alkyl and C₁-C₃-alkylthio-C₁-C₃-alkyl. More preferably still, R¹ is hydrogen, methyl, cyclopropyl, methoxy, ethoxy, -S-methyl, methylsulfonyl, and methoxyethoxy-. Most preferably R¹ is hydrogen.

Preferably R² is hydrogen or selected from the group consisting of: C₁-C₆-alkyl, C₂-C₆-alkenyl, C₃-C₆-alkynyl, C₁-C₆-alkoxy-C₁-C₆-alkyl, C₁-C₆alkoxy-C₁-C₆alkoxy, C₁-C₆-alkylthio-C₁-C₆-alkyl, C₃-C₆-cycloalkyl-C₁-C₆-alkyl, and cyano-C₁-C₆-alkyl, each of which is optionally substituted by 1-3 halogen atoms; or optionally substituted phenyl or optionally substituted benzyl, wherein said optional substituents are 1-3 substituents independently selected from halogen, C₁-C₆alkyl, and C₁-C₆alkoxy. More preferably R² is selected from C₁-C₃-alkyl, C₁-C₃-haloalkyl comprising no more than 3 halogen atoms, C₂-C₄-alkenyl, C₃-C₄-alkynyl, cyano-C₁-C₃-alkyl, C₁-C₃-alkylthio-C₁-C₃-alkyl. More preferably still, R² is methyl, ethyl, isopropyl, allyl, propargyl, difluoroethyl, trifluoroethyl, cyanomethyl, methoxyethyl, or methylthioethyl. Even more preferably R² is methyl, difluoroethyl, trifluoroethyl, or propargyl, yet more preferably R² is methyl, difluoroethyl or propargyl, and most preferably R² is 2,2-difluoroethyl or propargyl.

Preferably, G is hydrogen or —C(O)—R³, and R³ is selected from the group consisting of C₁-C₆alkyl, C₂-C₆alkenyl, C₂-C₆alkynyl, C₁-C₆alkyl-S—, C₁-C₆alkoxy, —NR⁵R⁶ and phenyl optionally substituted by one or more R⁷.

As defined herein, R⁵ and R⁶ are independently selected from the group consisting of C₁-C₆ alkyl, C₁-C₆ alkoxy-; or they can together form a morpholinyl ring. Preferably R⁵ and R⁶ are independently selected from the group consisting of C₁-C₄ alkyl, C₁-C₄ alkoxy-; or they can together form a morpholinyl ring. More preferably R⁵ and R⁶ are each independently selected from the group consisting of methyl, ethyl, propyl, methoxy, ethoxy and propoxy.

Preferably each R⁷ is independently selected from the group consisting of halogen, cyano, nitro, C₁-C₃alkyl, C₁-C₃haloalkyl, C₁-C₃alkoxy and C₁-C₃haloalkoxy.

Preferably R³ is C₁-C₄ alkyl, C₂-C₃alkenyl, C₂-C₃alkynyl,-C₁-C₃alkoxy, or —NR⁵R⁶ wherein R⁵ and R⁶ together form a morpholinyl ring. More preferably R³ is isopropyl, t-butyl, methyl, ethyl, propargyl or methoxy.

In one set of embodiments G is hydrogen or —C(O)—R³′ wherein R³ is C₁-C₄ alkyl, C₂-C₃alkenyl, C₂-C₃alkynyl or —C₁-C₃alkoxy. In a further set of embodiments G is hydrogen or —C(O)—R³′ wherein R³ is isopropyl, t-butyl, methyl, ethyl, propargyl or methoxy. However, it is particularly preferred that G is hydrogen.

X is preferably hydrogen, halogen, or C₁-C₃ haloalkyl; more preferably hydrogen, fluorine, chlorine, bromine, or C₁-C₂ haloalkyl. More preferably still, hydrogen, fluorine, chlorine, bromine or C₁ haloalkyl (such as trifluoromethyl).

In one set of embodiments it is preferred that X is ortho with respect to the bi-cyclic moiety. It is particularly preferred that X is either fluorine or chlorine and is ortho with respect to the thiazalopyridinone moiety.

Y is preferably hydrogen, C₁-C₃ alkyl, C₁-C₃haloalkyl, or halogen. In certain embodiments Y is preferably bromine or chlorine.

In one set of embodiments it is preferred that Y is ortho with respect to the benzyloxy moiety. It is particularly preferred that Y is ortho with respect to the benzyloxy moiety and is halogen, in particular chlorine.

As described herein, Z may be C₁-C₃ alkyl, C₁-C₃ alkoxy, C₁-C₃haloalkyl, C₁-C₃haloalkoxy, or halogen and n is an integer of 0, 1, 2, 3, 4, or 5. Accordingly, the benzyl moiety of formula (I) may be represented as follows wherein p denotes the point of attachment to the remainder of the molecule via the ether link:

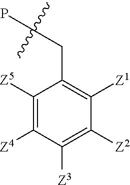

Preferably each Z radical is independently selected from halogen (in particular chlorine), methyl, methoxy, and trifluoromethoxy.

It is preferred that n is 0, 1, or 2. Where n is 1, it is preferred that Z is para with respect to the methoxy linker (i.e. Z is at position Z³). Where n is 2, it is preferred that one substituent will be para and the other will be meta with respect to the methoxy linker (i.e. one Z radical will be at position Z² or Z⁴, and the other Z radical will be at position Z³).

The compounds of Formula (I) according to the invention can be used as herbicides by themselves, but they are generally formulated into herbicidal compositions using formulation adjuvants, such as carriers, solvents and surface-active agents (SFAs). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of Formula (I).

Dustable powders (DP) may be prepared by mixing a compound of Formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of Formula (I) is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of Formula (I)).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The composition of the present may further comprise at least one additional pesticide. For example, the compounds according to the invention can also be used in combination with other herbicides or plant growth regulators. In a preferred embodiment the additional pesticide is a herbicide and/or herbicide safener. Examples of such mixtures are (in which ‘I’ represents a compound of Formula (I)). I+acetochlor, I+acifluorfen, I+acifluorfen-sodium, I+aclonifen, I+acrolein, I+alachlor, I+alloxydim, I+ametryn, I+amicarbazone, I+amidosulfuron, I+aminopyralid, I+amitrole, I+anilofos, I+asulam, I+atrazine, I+azafenidin, I+azimsulfuron, I+BCPC, I+beflubutamid, I+benazolin, I+bencarbazone, I+benfluralin, I+benfuresate, I+bensulfuron, I+bensulfuron-methyl, I+bensulide, I+bentazone, I+benzfendizone, I+benzobicyclon, I+benzofenap, I+bicyclopyrone, I+bifenox, I+bilanafos, I+bispyribac, I+bispyribac-sodium, I+borax, I+bromacil, I+bromobutide, I+bromoxynil, I+butachlor, I+butamifos, I+butralin, I+butroxydim, I+butylate, I+cacodylic acid, I+calcium chlorate, I+cafenstrole, I+carbetamide, I+carfentrazone, I+carfentrazone-ethyl, I+chlorflurenol, I+chlorflurenol-methyl, I+chloridazon, I+chlorimuron, I+chlorimuron-ethyl, I+chloroacetic acid, I+chlorotoluron, I+chlorpropham, I+chlorsulfuron, I+chlorthal, I+chlorthal-dimethyl, I+cinidon-ethyl, I+cinmethylin, I+cinosulfuron, I+cisanilide, I+clethodim, I+clodinafop, I+clodinafop-propargyl, I+clomazone, I+clomeprop, I+clopyralid, I+cloransulam, I+cloransulam-methyl, I+cyanazine, I+cycloate, I+cyclosulfamuron, I+cycloxydim, I+cyhalofop, I+cyhalofop-butyl, I+2,4-D, I+daimuron, I+dalapon, I+dazomet, I+2,4-DB, I+I+desmedipham, I+dicamba, I+dichlobenil, I+dichlorprop, I+dichlorprop-P, I+diclofop, I+diclofop-methyl, I+diclosulam, I+difenzoquat, I+difenzoquat metilsulfate, I+diflufenican, I+diflufenzopyr, I+dimefuron, I+dimepiperate, I+dimethachlor, I+dimethametryn, I+dimethenamid, I+dimethenamid-P, I+dimethipin, I+dimethylarsinic acid, I+dinitramine, I+dinoterb, I+diphenamid, I+dipropetryn, I+diquat, I+diquat dibromide, I+dithiopyr, I+diuron, I+endothal, I+EPTC, I+esprocarb, I+ethalfluralin, I+ethametsulfuron, I+ethametsulfuron-methyl, I+ethephon, I+ethofumesate, I+ethoxyfen, I+ethoxysulfuron, I+etobenzanid, I+fenoxaprop-P, I+fenoxaprop-P-ethyl, I+fentrazamide, I+ferrous sulfate, I+flamprop-M, I+flazasulfuron, I+florasulam, I+fluazifop, I+fluazifop-butyl, I+fluazifop-P, I+fluazifop-P-butyl, I+fluazolate, I+flucarbazone, I+flucarbazone-sodium, I+flucetosulfuron, I+fluchloralin, I+flufenacet, I+flufenpyr, I+flufenpyr-ethyl, I+flumetralin, I+flumetsulam, I+flumiclorac, I+flumiclorac-pentyl, I+flumioxazin, I+flumipropin, I+fluometuron, I+fluoroglycofen, I+fluoroglycofen-ethyl, I+fluoxaprop, I+flupoxam, I+flupropacil, I+flupropanate, I+flupyrsulfuron, I+flupyrsulfuron-methyl-sodium, I+flurenol, I+fluridone, I+flurochloridone, I+fluroxypyr, I+flurtamone, I+fluthiacet, I+fluthiacet-methyl, I+fomesafen, I+foramsulfuron, I+fosamine, I+glufosinate, I+glufosinate-ammonium, I+glyphosate, I+halauxifen, I+halosulfuron, I+halosulfuron-methyl, I+haloxyfop, I+haloxyfop-P, I+hexazinone, I+imazamethabenz, I+imazamethabenz-methyl, I+imazamox, I+imazapic, I+imazapyr, I+imazaquin, I+imazethapyr, I+imazosulfuron, I+indanofan, I+indaziflam, I+iodomethane, I+iodosulfuron, I+iodosulfuron-methyl-sodium, I+ioxynil, I+isoproturon, I+isouron, I+isoxaben, I+isoxachlortole, I+isoxaflutole, I+isoxapyrifop, I+karbutilate, I+lactofen, I+lenacil, I+linuron, I+mecoprop, I+mecoprop-P, I+mefenacet, I+mefluidide, I+mesosulfuron, I+mesosulfuron-methyl, I+mesotrione, I+metam, I+metamifop, I+metamitron, I+metazachlor, I+methabenzthiazuron, I+methazole, I+methylarsonic acid, I+methyldymron, I+methyl isothiocyanate, I+metolachlor, I+S-metolachlor, I+metosulam, I+metoxuron, I+metribuzin, I+metsulfuron, I+metsulfuron-methyl, I+molinate, I+monolinuron, I+naproanilide, I+napropamide, I+naptalam, I+neburon, I+nicosulfuron, I+n-methyl glyphosate, I+nonanoic acid, I+norflurazon, I+oleic acid (fatty acids), I+orbencarb, I+orthosulfamuron, I+oryzalin, I+oxadiargyl, I+oxadiazon, I+oxasulfuron, I+oxaziclomefone, I+oxyfluorfen, I+paraquat, I+paraquat dichloride, I+pebulate, I+pendimethalin, I+penoxsulam, I+pentachlorophenol, I+pentanochlor, I+pentoxazone, I+pethoxamid, I+phenmedipham, I+picloram, I+picolinafen, I+pinoxaden, I+piperophos, I+pretilachlor, I+primisulfuron, I+primisulfuron-methyl, I+prodiamine, I+profoxydim, I+prohexadione-calcium, I+prometon, I+prometryn, I+propachlor, I+propanil, I+propaquizafop, I+propazine, I+propham, I+propisochlor, I+propoxycarbazone, I+propoxycarbazone-sodium, I+propyzamide, I+prosulfocarb, I+prosulfuron, I+pyraclonil, I+pyraflufen, I+pyraflufen-ethyl, I+pyrasulfotole, I+pyrazolynate, I+pyrazosulfuron, I+pyrazosulfuron-ethyl, I+pyrazoxyfen, I+pyribenzoxim, I+pyributicarb, I+pyridafol, I+pyridate, I+pyriftalid, I+pyriminobac, I+pyriminobac-methyl, I+pyrimisulfan, I+pyrithiobac, I+pyrithiobac-sodium, I+pyroxasulfone, I+pyroxsulam, I+quinclorac, I+quinmerac, I+quinoclamine, I+quizalofop, I+quizalofop-P, I+rimsulfuron, I+saflufenacil, I+sethoxydim, I+siduron, I+simazine, I+simetryn, I+sodium chlorate, I+sulcotrione, I+sulfentrazone, I+sulfometuron, I+sulfometuron-methyl, I+sulfosate, I+sulfosulfuron, I+sulfuric acid, I+tebuthiuron, I+tefuryltrione, I+tembotrione, I+tepraloxydim, I+terbacil, I+terbumeton, I+terbuthylazine, I+terbutryn, I+thenylchlor, I+thiazopyr, I+thifensulfuron, I+thiencarbazone, I+thifensulfuron-methyl, I+thiobencarb, I+topramezone, I+tralkoxydim, I+tri-allate, I+triasulfuron, I+triaziflam, I+tribenuron, I+tribenuron-methyl, I+triclopyr, I+trietazine, I+trifloxysulfuron, I+trifloxysulfuron-sodium, I+trifluralin, I+triflusulfuron, I+triflusulfuron-methyl, I+trihydroxytriazine, I+trinexapac-ethyl, I+tritosulfuron, I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6). The compounds of the present invention may also be combined with herbicidal compounds disclosed in WO06/024820 and/or WO07/096576.

The mixing partners of the compound of Formula (I) may also be in the form of esters or salts, as mentioned e.g. in The Pesticide Manual, Fourteenth Edition, British Crop Protection Council, 2006.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula I with the mixing partner).

The compounds of Formula (I) according to the invention can also be used in combination with one or more safeners. Likewise, mixtures of a compound of Formula (I) according to the invention with one or more further herbicides can also be used in combination with one or more safeners. The safeners can be AD 67 (MON 4660), benoxacor, cloquintocet-mexyl, cyprosulfamide (CAS RN 221667-31-8), dichlormid, fenchlorazole-ethyl, fenclorim, fluxofenim, furilazole and the corresponding R isomer, isoxadifen-ethyl, mefenpyr-diethyl, oxabetrinil, N-isopropyl-4-(2-methoxy-benzoylsulfamoyl)-benzamide (CAS RN 221668-34-4). Other possibilities include safener compounds disclosed in, for example, EP0365484 e.g N-(2-methoxybenzoyl)-4-[(methylaminocarbonyl)amino]benzenesulfonamide. Particularly preferred are mixtures of a compound of Formula I with cyprosulfamide, isoxadifen-ethyl, cloquintocet-mexyl and/or N-(2-methoxybenzoyI)-4-[(methyl-aminocarbonyl)amino]benzenesulfonamide.

The safeners of the compound of Formula I may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of Formula (I) to safener is from 100:1 to 1:10, especially from 20:1 to 1:1.

The mixtures can advantageously be used in the above-mentioned formulations (in which case “active ingredient” relates to the respective mixture of compound of Formula I with the safener).

The present invention still further provides a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of Formula (I) may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®. In a particularly preferred aspect, the crop plant has been engineered to over-express homogentisate solanesyltransferase as taught in, for example, WO2010/029311.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

The compositions can be used to control unwanted plants (collectively, ‘weeds’). The weeds to be controlled include both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium. Weeds can also include plants which may be considered crop plants but which are growing outside a crop area (‘escapes’), or which grow from seed left over from a previous planting of a different crop (‘volunteers’). Such volunteers or escapes may be tolerant to certain other herbicides.

The compounds of the present invention may be prepared according to the following schemes, in which the substituents R¹, R², G, R³, X, Y, Z, and n have (unless otherwise stated explicitly) the definitions described hereinbefore. Certain compounds (1a) of the present invention may be prepared from compounds of formula (2) as shown in Reaction scheme 1.

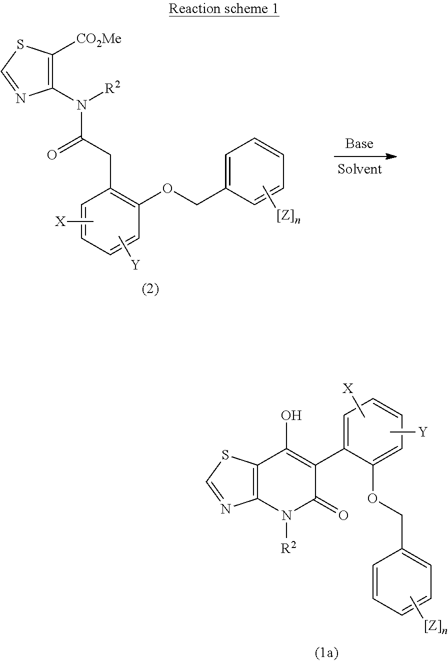

Compounds of formula (1a) may be prepared by treatment of amide compounds (2) with a suitable base in a suitable solvent at a temperature between −10 and 50° C. Examples of suitable bases are NaH or LiHMDS, and of suitable solvents are tetrahydrofuran [THF] or N,N-dimethylformamide [DMF].

Compounds of formula (2) may be prepared from compounds of formula (3) as shown in Reaction scheme 2.

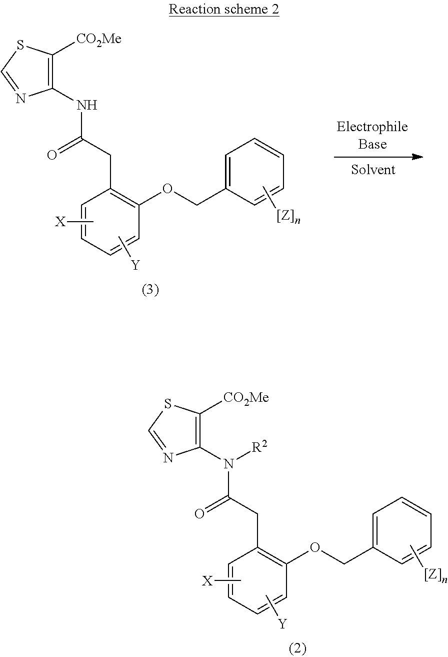

Compounds of formula (2) may be prepared by N-alkylation of compounds (3) with an electrophilic alkylating agent, in the presence of a suitable base and solvent, at a temperature between 0 and 100° C. Examples of suitable electrophile reagents are propargyl bromide, methyl iodide, dimethyl sulfate or 2,2-difluoroethyl triflate. Examples of suitable bases are NaH, diisopropylethylamine or potassium carbonate, and of suitable solvents are THF or acetonitrile.

Compounds of formula (3) may be prepared by amide coupling of amino-thiazole (5) with phenylacetic acids (4) as shown in Reaction scheme 3.

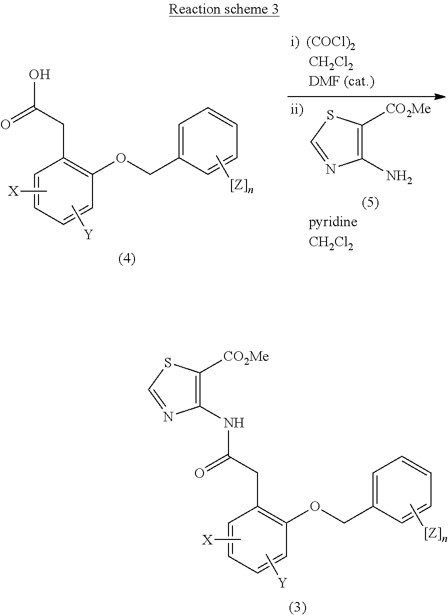

Amino-thiazole (5) is prepared according to Reaction scheme 4 (a method which is known from PCT patent application WO2012/087976). Cyanimidodithiocarbonic acid monomethyl ester monopotassium salt is readily available commercially.

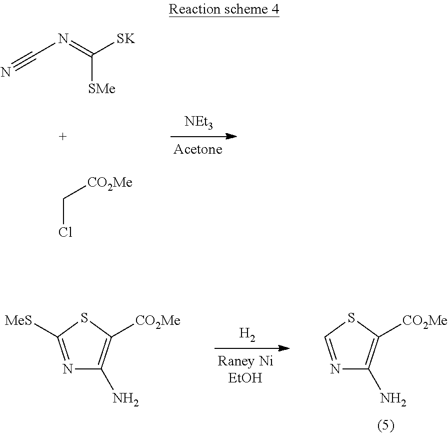

Compounds of formula (4) may be prepared by oxidation of olefins (6) according to Reaction scheme 5.

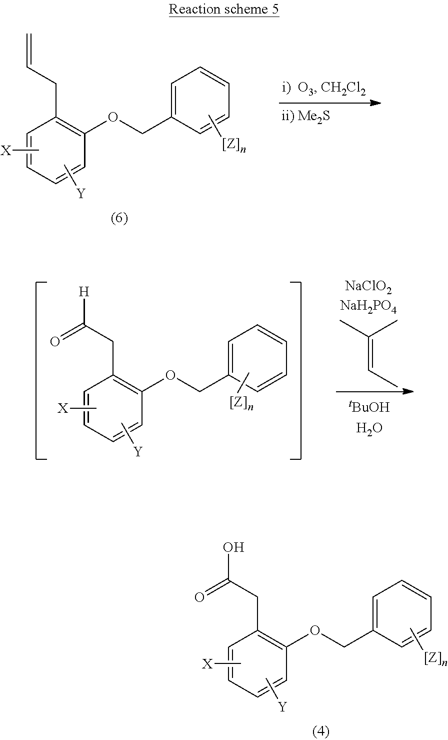

Certain olefin compounds (6), specifically subset (6a), may be prepared by the O-alkylation of 2-allyl-phenols (7) with a benzyl halide compound, as shown in Reaction scheme 6.

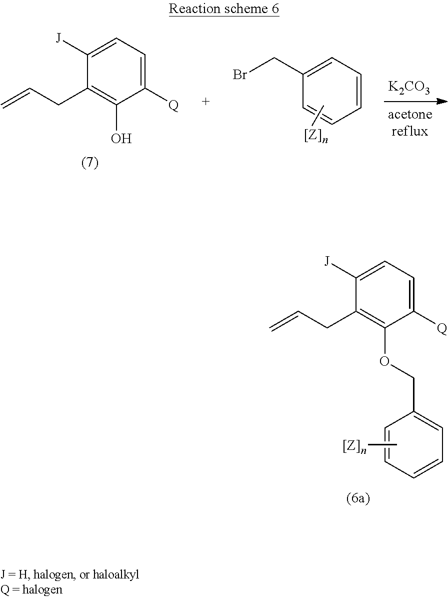

With reference to Reaction scheme 6, many benzyl halides are commercially available. An example is benzyl bromide. 2-allyl-phenols (7) may be prepared as shown in Reaction scheme 7.

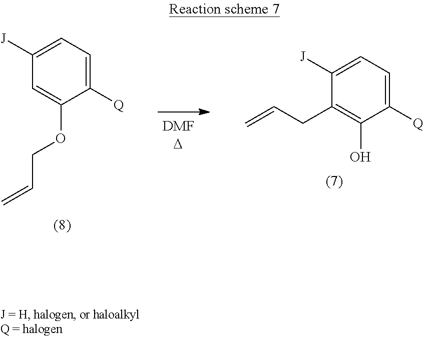

With reference to Reaction scheme 7, an example of compounds (8) is 2-allyloxy-1,4-dichloro-benzene, prepared according to J. Chem. Soc., Perkin Trans. 2, 2001, 1824. Other compounds (8) may be synthesised similarly, according to Reaction scheme 8.

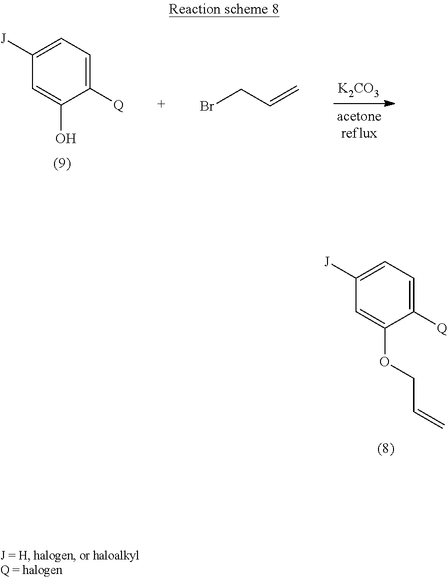

With reference to Reaction scheme 8, many phenol compounds (9) are commercially available. An example is 2,5-dichlorophenol.

Various aspects and embodiments of the present invention will now be illustrated in more detail by way of example. It will be appreciated that modification of detail may be made without departing from the scope of the invention.

### PREPARATION EXAMPLES

### Example 1

### Preparation of 6-(2-benzyloxy-3,6-dichloro-phenyl)-7-hydroxy-4-prop-2-ynyl-thiazolo[4,5-b]pyridin-5-one

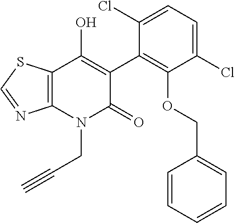 [SMILES: C#CCn1c(=O)c(-c2c(Cl)ccc(Cl)c2OCc2ccccc2)c(O)c2scnc21]

1.1 2-Allyl-3,6-dichloro-phenol

A mixture of 2-allyloxy-1,4-dichloro-benzene (1.0 g, 4.9 mmol, 1.0 eq.) and DMF (0.1 mL) was heated at an external temperature of 220° C. for 1 hour. The mixture was allowed to cool to room temperature and was concentrated in vacuo to provide 2-allyl-3,6-dichloro-phenol as a brown oil (0.99 g, 99%).

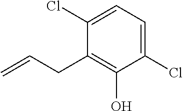

¹H NMR (400 MHz, CDCl₃): δ_{H}: 7.18-7.08 (1H, m) 6.95-6.85 (1H, m) 6.02-5.84 (1H, m) 5.71 (1 H, s) 5.14-4.99 (2H, m) 3.59 (2H, dt).
1.2 2-Allyl-3-benzyloxy-1,4-dichloro-benzene

Benzyl bromide (3.2 mL, 27 mmol, 1.1 eq.) was added to a suspension of 2-allyl-3,6-dichloro-phenol (5.0 g, 25 mmol, 1.0 eq.) and potassium carbonate (3.7 g, 27 mmol, 1.1 eq.) in acetone (49 mL) and the mixture was heated at reflux for 6 hours. The mixture was allowed to cool to room temperature and was filtered. The filtrate was concentrated in vacuo and the crude product was purified by flash column chromatography to provide 2-allyl-3-benzyloxy-1,4-dichloro-benzene (4.031 g, 56%) as a colourless oil.

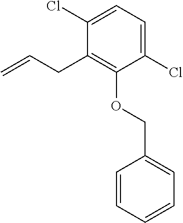 [SMILES: C=CCc1c(Cl)ccc(Cl)c1OCc1ccccc1]

¹H NMR (400 MHz, CDCl₃): δ_{H}: 7.54-7.49 (2H, m), 7.45-7.35 (3H, m), 7.27-7.24 (1H, m), 7.15 (1H, d), 6.01-5.90 (1H, m), 5.10-4.97 (4H, m), 3.59 (2H, dt).

1.3 2-(2-Benzyloxy-3,6-dichloro-phenyl)acetic acid

A solution of 2-allyl-3-benzyloxy-1,4-dichloro-benzene (38.1 g, 130 mmol, 1.00 eq.) in dichloromethane (650 mL) in a 3-necked flask was cooled to −78° C. One side neck was connected to a Dreshel bottle containing an aqueous solution of Kl (100 mL, 15% w/w). Ozone was bubbled through the solution until 2-allyl-3-benzyloxy-1,4-dichloro-benzene had been fully consumed (4 hours). Air was bubbled through the solution for 10 minutes to remove excess ozone. The bubbling of gas through the solution was stopped and dimethyl sulfide (95.4 mL, 1300 mmol, 10.0 eq.) was added. The mixture was allowed to warm to room temperature and was stirred for 12 hours. The mixture was washed with brine (2×200 mL) and the organic extracts were passed through a hydrophobic frit. The mixture was concentrated in vacuo to provide a yellow oil (43 g). The residue was dissolved in a mixture of tert-butanol (260 mL) and water (130 mL) then cooled to 0° C. 2-methylbut-2-ene (135 mL, 1300 mmol, 10.0 eq.), sodium dihydrogen phosphate (62.4 g, 520 mmol, 4.00 eq.) and sodium chlorite (44.1 g, 390 mmol, 3.00 eq.) were added. The mixture was stirred for 2 hours then diluted with brine (300 mL) and 2.0M hydrochloric acid (300 mL). The mixture was extracted with EtOAc (3×200 mL). The combined organic extracts were washed with a saturated aqueous solution of sodium metabisulfite (200 mL) then passed through a hydrophobic frit and concentrated in vacuo to provide a pale yellow solid (41.4 g). The residue was suspended in H₂O (200 mL) and an aqueous solution of NaOH (30 mL, 2.0 M) was added resulting in a clear solution. The mixture was washed with Et₂O (100 mL) and the aqueous layer was acidified by addition of concentrated HCl (20 mL) resulting in the formation of a precipitate. The mixture was filtered and the filtrand was dried in vacuo to provide 2-(2-benzyloxy-3,6-dichloro-phenyl)acetic acid (29.2 g, 72%) as a white solid.

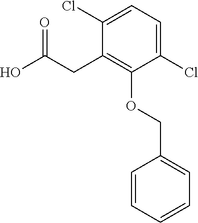 [SMILES: O=C(O)Cc1c(Cl)ccc(Cl)c1OCc1ccccc1]

¹H NMR (400 MHz, CDCl₃): δ_{H}: 7.48-7.44 (2H, m), 7.42-7.31 (4H, m), 7.17 (1H, d), 5.04 (2H, s), 3.85 (2H, s).
1.4 methyl 4-[[2-(2-benzyloxy-3,6-dichloro-phenyl)acetyl]amino]thiazole-5-carboxylate

To a stirred solution of 2-(2-benzyloxy-3,6-dichloro-phenyl)acetic acid (1.50 g, 4.82 mmol) in dichloromethane (15 mL) was added DMF (0.05 mL). The mixture was cooled in an ice/water bath. Oxalyl chloride (0.83 mL, 9.64 mmol) was added dropwise and the reaction mixture was then stirred at ambient temperature for 1.5 h. GC/MS analysis (after quenching an aliquot into methanol) showed starting material consumed and acid chloride formed (detected as its methyl ester derivative). The reaction mixture was evaporated to dryness and redissolved in dichloromethane (7.5 mL).

Separately, methyl 4-aminothiazole-5-carboxylate (572 mg, 3.62 mmol) was dissolved in a mixture of dichloromethane (15 mL) and pyridine (0.97 mL, 12.05 mmol) and cooled to 0° C. The acid chloride solution was added dropwise to the aminothiazole, maintaining the reaction at 0° C. After completion of the addition, the reaction mixture was stirred at ambient temperature for a further 2 h. It was then diluted with dichloromethane and washed sequentially with 1N HCl, saturated NaHCO₃ solution, water and brine. The organics were dried over Na₂SO₄, filtered and evaporated to dryness. Purification by flash column chromatography gave crude target compound which was triturated with 20%v/v diethyl ether in hexane to obtain methyl 4-[[2-(2-benzyloxy-3,6-dichloro-phenypacetyl]amino]thiazole-5-carboxylate (1.1 g, 51%) as an off-white solid.

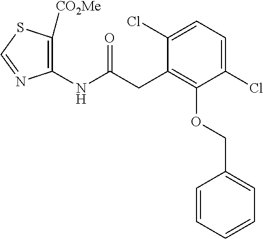 [SMILES: COC(=O)c1scnc1NC(=O)Cc1c(Cl)ccc(Cl)c1OCc1ccccc1]

¹H NMR (400 MHz, CDCl₃): δ_{H}: 9.8 (bs, 1H), 8.8 (s, 1H), 7.45 (d, J=6.8, 2H), 7.35-7.28 (m, 4H), 7.19 (d, J=8.4, 1H), 5.09 (s, 2H), 4.11 (s, 2H), 3.85 (s, 3H).

1.5 methyl 4-[[2-(2-benzyloxy-3,6-dichloro-phenyl)acetyl]-prop-2-ynyl-amino]thiazole-5-carboxylate

To a stirred solution of methyl 4-[[2-(2-benzyloxy-3,6-dichloro-phenyl)acetyl]amino]thiazole-5-carboxylate (1.1 g, 2.44 mmol) in acetonitrile (25 mL) at ambient temperature was added K₂OC₃ (842 mg, 6.1 mmol) then propargyl bromide (0.46 mL, 6.1 mmol). The reaction mixture was refluxed for 10 h then allowed to cool.

The mixture was diluted with water and ethyl acetate, the organic layer separated and kept, and the aqueous re-extracted with further ethyl acetate. The combined organics were washed with brine, dried over Na₂SO₄, filtered and evaporated to obtain a crude residue. Flash column chromatography gave methyl 4-[[2-(2-benzyloxy-3,6-dichloro-phenyl)acetyl]-prop-2-ynyl-amino]thiazole-5-carboxylate (650 mg, 54%) as a yellow oil.

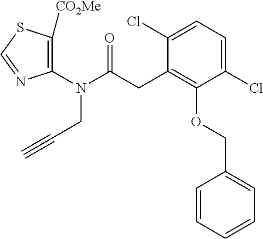 [SMILES: C#CCN(C(=O)Cc1c(Cl)ccc(Cl)c1OCc1ccccc1)c1ncsc1C(=O)OC]

¹H NMR (400 MHz, CDCl₃): δ_{H}: 8.80 (s, 1H), 7.51 (d, J=7.0, 2H), 7.40-7.33 (m, 3H), 7.22 (s, 1H), 7.09 (d, J=8.6, 1H), 4.96 (s, 2H), 4.64 (s, J=2.3, 2H), 3.74 (s, 3H), 3.70 (s, 2H).
1.6 6-(2-benzyloxy-3,6-dichloro-phenyl)-7-hydroxy-4-prop-2-ynyl-thiazolo[4,5-b]pyridin-5-one

To a stirred solution of methyl 4-[[2-(2-benzyloxy-3,6-dichloro-phenyl)acetyl]-prop-2-ynyl-amino]thiazole-5-carboxylate (550 mg, 1.12 mmol) in N,N-dimethylformamide (5 mL) at 0° C. was added sodium hydride (60% dispersed in oil, 50 mg, 1.4 mmol). The reaction mixture was then stirred at ambient temperature for 1 h. TLC analysis showed consumption of starting material. The mixture was quenched with 2N HCl and extracted with ethyl acetate. The organic layer was dried over Na₂SO₄, evaporated to dryness and the crude purified by flash column chromatography to give 6-(2-benzyloxy-3,6-dichloro-phenyl)-7-hydroxy-4-prop-2-ynyl-thiazolo[4,5-b]pyridin-5-one (300 mg, 58%) as an off-white solid.

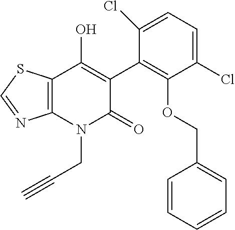 [SMILES: C#CCn1c(=O)c(-c2c(Cl)ccc(Cl)c2OCc2ccccc2)c(O)c2scnc21]

¹H NMR (400 MHz, CDCl₃): δ_{H}: 11.46 (s, 1H), 9.40 (s, 1H), 7.58 (d, J=8.8, 1H), 7.39 (d, J=8.8, 1H), 7.14-7.09 (m, 5H), 5.01 (s, 2H), 4.84-4.76 (m, 2H), 3.16 (s, 1H).

Table 1 below provides 30 specific examples of compounds of formula (I) of the invention, wherein R¹ is H, G is H, and n is 0, and R², X and Y are as shown in the table. Compounds 1.001, 1.007, 1.009, 1.011, 1.017, 1.019, 1.021, 1.027 and 1.029 were prepared using the general methods as described supra.

TABLE 1
Examples of herbicidal compounds of the present invention.
(Ia)

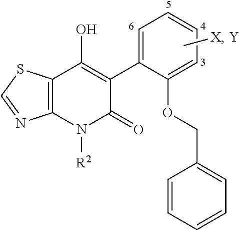

In this formula (Ia), which represents compounds of formula (I)
wherein R¹ is H, G is H, and n is 0, the numbering system used to denote
the position of X and Y is shown for the purposes of clarity.

| Com- | | | | |
|---|---|---|---|---|
| pound | R² | X | Y | NMR |
| 1.001 | —CH₂C≡CH | 6—Cl | 3—Cl | ¹H NMR (400 MHz, d6-DMSO) δ_{H}: |
| | | | | 11.46 (1H, bs), 9.40 (1H, s), |
| | | | | 7.58 (1H, d), 7.40 (1H, d), 7.17- |
| | | | | 7.05 (5H, m), 5.07-4.95 (2H, m), |
| | | | | 4.86-4.74 (2H, m), 3.17 (1H, s). |
| 1.002 | —CH₂C≡CH | 6—Cl | 3—Br | |
| 1.003 | —CH₂C≡CH | 6—Br | 3—Cl | |
| 1.004 | —CH₂C≡CH | 6—Br | 3—Br | |
| 1.005 | —CH₂C≡CH | H | 3—Cl | |
| 1.006 | —CH₂C≡CH | H | 3—Br | |
| 1.007 | —CH₂C≡CH | 6—F | 3—Cl | ¹H NMR (400 MHz, d6-DMSO) δ_{H}: |
| | | | | 11.53 (1H, bs), 9.41 (1H, s), 7.62- |
| | | | | 7.56 (1H, m), 7.18-7.07 (5H, m), |
| | | | | 5.10-4.90 (2H, m), 4.90-4.75 |
| | | | | (2H, m), 3.18 (1H, s). |
| 1.008 | —CH₂C≡CH | 6—F | 3—Br | |
| 1.009 | —CH₂C≡CH | 6—CF₃ | 3—Cl | |
| 1.010 | —CH₂C≡CH | 6—CF₃ | 3—Br | |
| 1.011 | —Me | 6—Cl | 3—Cl | ¹H NMR (400 MHz, d6-DMSO) δ_{H}: |
| | | | | 11.25 (1H, s), 9.36 (1H, s), 7.57 |
| | | | | (1H, d), 7.39 (1H, d), 7.17-6.99 |
| | | | | (5H, m), 4.89-4.73 (2H, m), 3.62 |
| | | | | (3H, s). |
| 1.012 | —Me | 6—Cl | 3—Br | |
| 1.013 | —Me | 6—Br | 3—Cl | |
| 1.014 | —Me | 6—Br | 3—Br | |
| 1.015 | —Me | H | 3—Cl | |
| 1.016 | —Me | H | 3—Br | |
| 1.017 | —Me | 6—F | 3—Cl | ¹H NMR (400 MHz, d6-DMSO) δ_{H}: |
| | | | | 11.32 (1H, bs), 9.36 (1H, s), 7.60- |
| | | | | 7.54 (1H, m), 7.17-7.04 (6H, m), |
| | | | | 4.90-4.72 (2H, m), 3.62 (3H, s). |
| 1.018 | —Me | 6—F | 3—Br | |
| 1.019 | —Me | 6—CF₃ | 3—Cl | ¹H NMR (400 MHz, d6-DMSO) δ_{H}: |
| | | | | 11.25 (1H, s), 9.35 (1H, s), |
| | | | | 7.79 (1H, d), 7.63 (1H, d), |
| | | | | 7.15-7.06 (3H, m), 7.03-6.98 |
| | | | | (2H, m), 4.94-4.82 (2H, m), |
| | | | | 3.58 (3H, s). |
| 1.020 | —Me | 6—CF₃ | 3—Br | |
| 1.021 | CH₂CHF₂ | 6—Cl | 3—Cl | ¹H NMR (400 MHz, d6-DMSO) δ_{H}: |
| | | | | 11.58 (1H, bs), 9.39 (1H, s), |
| | | | | 7.58 (1H, d), 7.39 (1H, d), |
| | | | | 7.19-7.03 (5H, m), 6.27 (1H, tt), |
| | | | | 4.85-4.73 (2H, m), 4.73-4.61 |
| | | | | (2H, m). |
| 1.022 | CH₂CHF₂ | 6—Cl | 3—Br | |
| 1.023 | CH₂CHF₂ | 6—Br | 3—Cl | |
| 1.024 | CH₂CHF₂ | 6—Br | 3—Br | |
| 1.025 | CH₂CHF₂ | H | 3—Cl | |
| 1.026 | CH₂CHF₂ | H | 3—Br | |
| 1.027 | CH₂CHF₂ | 6—F | 3—Cl | ¹H NMR (400 MHz, d6-DMSO) δ_{H}: |
| | | | | 11.65 (1H, bs), 9.40 (1H, s), |
| | | | | 7.63-7.56 (1H, m), 7.19-7.04 |
| | | | | (6H, m), 6.28 (1H, tt), 4.88- |
| | | | | 4.73 (2H, m), 4.73-4.62 (2H, m). |
| 1.028 | CH₂CHF₂ | 6—F | 3—Br | |
| 1.029 | CH₂CHF₂ | 6—CF₃ | 3—Cl | ¹H NMR (400 MHz, d6-DMSO) δ_{H}: |
| | | | | 11.59 (1H, bs), 9.38 (1H, s), |
| | | | | 7.80 (1H, d), 7.64 (1H, d), 7.17- |
| | | | | 6.99 (5H, m), 6.23 (1H, tt), |
| | | | | 4.86 (2H, s), 4.70-4.58 (2H, m). |
| 1.030 | CH₂CHF₂ | 6—CF₃ | 3—Br | |

### BIOLOGICAL EXAMPLES

### B1 Post-Emergence Efficacy

Seeds of a variety of test species (such as, for example, Solanum nigrum (SOLNI), Amaranthus retoflexus (AMARE), Setaria faberi (SETFA), Echinochloa crusgalli (ECHCG), Ipomoea hederacea (IPOHE) and Lolium perenne (LOLPE)) are sown in standard soil in pots. After 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween 20 (polyoxyethylene sorbitan monolaurate, CAS RN 9005-64-5). Compounds are applied at 1000 and 62.5 g/ha. The test plants are then grown under controlled conditions in a glasshouse (at 24/16° C., day/night; 14 hours light; 65% humidity) and watered twice daily. After 13 days, the test is evaluated for the percentage damage caused to the plant. The biological activities are assessed on a five point scale (5=80-100%; 4=60-79%; 3=40-59%; 2=20-39%; 1=0-19%).

TABLE 2
Control of weed species by compounds of formula (I) after post-emergence
application.

| | Application | | | | | | |
|---|---|---|---|---|---|---|---|
| Compound | rate (g/ha) | LOLPE | SOLNI | AMARE | SETFA | ECHCG | IPOHE |
| 1.001 | 1000 | 5 | 5 | 5 | 5 | 5 | 5 |
| | 62.5 | 5 | 5 | 5 | 5 | 5 | 5 |
| 1.007 | 1000 | 5 | 5 | 5 | 5 | 5 | 5 |
| | 62.5 | 5 | 5 | 5 | 5 | 5 | 5 |
| 1.009 | 1000 | 5 | 5 | 5 | 5 | 5 | 5 |
| | 62.5 | 5 | 5 | 5 | 5 | 5 | 5 |
| 1.011 | 1000 | 5 | 5 | 5 | 5 | 5 | 5 |
| | 62.5 | 2 | 5 | 4 | 2 | 3 | 5 |
| 1.017 | 1000 | 5 | 5 | 5 | 5 | 5 | 5 |
| | 62.5 | 4 | 5 | 4 | 1 | 2 | 5 |
| 1.019 | 1000 | 5 | 5 | 5 | 5 | 5 | 5 |
| | 62.5 | 3 | 5 | 4 | 3 | 3 | 5 |
| 1.021 | 1000 | 5 | 5 | 5 | 5 | 5 | 5 |
| | 62.5 | 4 | 5 | 5 | 4 | 4 | 5 |
| 1.027 | 1000 | 5 | 5 | 5 | 5 | 5 | 5 |
| | 62.5 | 4 | 5 | 5 | 3 | 3 | 5 |
| 1.029 | 1000 | 5 | 5 | 5 | 5 | 5 | 5 |
| | 62.5 | 2 | 5 | 4 | 2 | 3 | 4 |

1. A compound of formula (I) 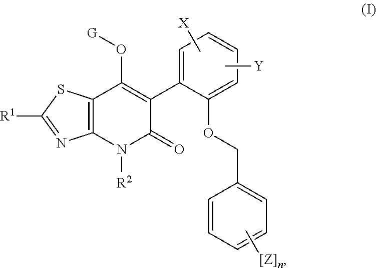
or a salt or N-oxide thereof,
wherein:
R¹ is hydrogen, halogen, nitro, cyano, or selected from the group consisting of : C₁-C₆alkyl, C₂-C₆ alkenyl, C₁-C₆ alkoxy C₁-C₆ alkylthio, C₁-C₆alkylsulfinyl, C₁-C₆alkylsulfonyl, C₃-C₆cycoalkyl, C₁-C₆ alkoxy-C₁-C₆alkyl-, C₁-C₆alkoxy-C₁-C₆alkoxy-, di-C₁-C₆alkoxy-C₁-C₆alkyl, and C₁-C₆alkylthio-C₁-C₆alkyl, each of which is optionally substituted by 1-3 halogen atoms;
R² is hydrogen; or C₁-C₆alkyl, C₂-C₆ alkenyl, C₃-C₆alkynyl C₃-C₆cycoalkyl, C₁-C₆ alkoxy-C₁-C₆alkyl-, C₁-C₆alkoxy-C₁-C₆alkoxy-, di-C₁-C₆alkoxy-C₁-C₆alkyl, C₁-C₆alkylthio-C₁-C₆alkyl, C₁-C₆alkylsulfinyl-C₁-C₆alkyl, C₁-C₆alkylsulfonyl-C₁-C₆alkyl, C₃-C₆cycoalkyl-C₁-C₆alkyl, or cyano-C₁-C₆alkyl, each of which is optionally substituted by 1-3 halogen atoms; or phenyl optionally substituted by 1-3 substituents independently selected from halogen, C₁-C₆ alkyl and C₁-C₆ alkoxy; or benzyl optionally substituted by 1-3 substituents independently selected from halogen, C₁-C₆ alkyl and C₁-C₆ alkoxy;
G is hydrogen, C₁-C₁₀alkyl, C₂-C₁₀alkenyl, C₂-C₁₀alkynyl or aryl-C₁-C₄alkyl- or aryl-C₁-C₄alkyl- wherein the aryl moiety is substituted by one to five substituents independently selected from halo, cyano, nitro, C₁-C₆alkyl, C₁-C₆haloalkyl or C₁-C₆alkoxy, or C(O)R^{3′};
R³ is C₁-C₁₀alkyl, C₃-C₁₀cycloalkyl, C₃-C₁₀cycloalkyl-C₁-C₁₀alkyl-, C₁-C₁₀haloalkyl, C₂-C₁₀alkenyl, C₂-C₁₀alkynyl, C₁-C₄alkoxy-C₁-C₁₀alkyl-, C₁-C₄alkylthio-C₁-C₄alkyl-, C₁-C₁₀alkoxy, C₂-C₁₀alkenyloxy, C₂-C₁₀alkynyloxy, C₁-C₁₀alkylthio-, NR⁵R⁶, N—C₁-C₄alkyl-amino-, N,N-di-(C₁-C₄alkyl)-amino-, aryl or aryl substituted by 1-3 R⁴, which may be the same or different, heteroaryl or heteroaryl substituted by 1-3 R⁴, which may be the same or different, aryl-C₁-C₄alkyl- or aryl-C₁-C₄alkyl- wherein the aryl moiety is substituted by 1-3 to three R⁴, which may be the same or different, heteroaryl-C₁-C₄alkyl- or heteroaryl-C₁-C₄alkyl- wherein the heteroaryl moiety is substituted by 1-3 R⁴, which may be the same or different, aryloxy- or aryloxy-substituted by 1-3 R⁴, which may be the same or different, heteroaryloxy- or heteroaryloxy-substituted by 1-3 R⁴, which may be the same or different, arylthio- or arylthio-substituted by 1-3 R⁴, which may be the same or different, or heteroarylthio- or heteroarylthio-substituted by one to three R⁴, which may be the same or different;
each R⁴ is independently halo, cyano, nitro, C₁-C₁₀alkyl, C₁-C₄haloalkyl, C₁-C₁₀alkoxy, C₁-C₄alkoxycarbonyl-, C₁-C₄haloalkoxy, C₁-C₁₀alkylthio-, C₁-C₄haloalkylthio-, C₁-C₁₀alkylsulfinyl-, C₁-C₄haloalkylsulfinyl-, C₁-C₁₀alkylsulfonyl-, C₁-C₄haloalkylsulfonyl-, aryl or aryl substituted by 1-5 substituents independently selected from halo, cyano, nitro, C₁-C₆alkyl, C₁-C₅haloalkyl or C₁-C₆alkoxy, or heteroaryl or heteroaryl substituted by 1-4 substituents independently selected from halo, cyano, nitro, C₁-C₆alkyl, C₁-C₆haloalkyl or C₁-C₆alkoxy;
R⁵ and R⁶ are independently selected from the group consisting of C₁-C₆ alkyl and C₁-C₆ alkoxy, or R⁵ and R⁶ together can form a morpolinyl ring;
X and Y are each independently hydrogen, C₁-C₃ alkyl, C₁-C₃ alkoxy, C₁-C₃haloalkyl, C₁-C₃haloalkoxy, or halogen,
Z is C₁-C₃ alkyl, C₁-C₃ alkoxy, C₁-C₃haloalkyl, C₁-C₃haloalkoxy, or halogen; and
n is an integer of 0, 1, 2, 3, 4, or 5.

2. The compound according to claim 1, wherein R¹ is hydrogen, or selected from the group consisting of: C₁-C₆-alkyl, C₁-C₆-alkoxy, C₁-C₆alkoxy-C₁-C₆alkoxy-, C₁-C₆-alkylthio, C₁-C₆-alkylsulfonyl, C₃-C₆-cycloalkyl, C₁-C₆-alkoxy-C₁-C₆-alkyl and C₁-C₆-alkylthio-C₁-C₆-alkyl, each of which is optionally substituted by 1-3 halogen atoms.

3. (canceled)

4. (canceled)

5. The compound according to claim 1, wherein R¹ is hydrogen.

6. The compound according to claim 1, wherein R² is hydrogen or selected from the group consisting of: C₁-C₆-alkyl, C₂-C₆-alkenyl, C₃-C₆-alkynyl, C₁-C₆-alkoxy-C₁-C₆-alkyl, C₁-C₆alkoxy-C₁-C₆alkoxy, C₁-C₆-alkylthio-C₁-C₆-alkyl, C₃-C₆-cycloalkyl-C₁-C₆-alkyl, and cyano-C₁-C₆-alkyl, each of which is optionally substituted by 1-3 halogen atoms; or optionally substituted phenyl or optionally substituted benzyl, wherein said optional substituents are 1-3 substituents independently selected from halogen, C₁-C₆alkyl, and C₁-C₆alkoxy.

7. (canceled)

8. (canceled)

9. The compound according to claim 1, wherein R¹ is hydrogen and R² is methyl, difluoroethyl, trifluoroethyl, or propargyl.

10. The compound according to claim 1 wherein X is hydrogen, halogen, or C₁-C₃haloalkyl.

11. (canceled)

12. The compound according to claim 1 wherein Y is hydrogen, C₁-C₃ alkyl, C₁-C₃haloalkyl, or halogen.

13. The compound according to claim 1 wherein n is 0, 1, or 2.

14. The compound according to claim 1 wherein each Z is independently selected from halogen, methyl, methoxy, and trifluoromethoxy.

15. The compound according to claim 1 wherein G is C(O)R³, and R³ is selected from the group consisting of C₁-C₆alkyl, C₁-C₆alkenyl, C₁-C₆alkynyl, C₁-C₆alkyl-S—, —NR⁴R⁵ and phenyl optionally substituted by one or more R⁷.

16. The compound according to claim 1 wherein G is C(O)NR⁴R⁵, and R⁴ and R⁵ are independently selected from the group consisting of C₁-C₆ alkyl and C₁-C₆ alkoxy, or R⁴ and R⁵ together can form a morpolinyl ring.

17. (canceled)

18. The compound according to claim 1, wherein G is hydrogen.

19. A herbicidal composition comprising a herbicidal compound according to claim 1 and an agriculturally acceptable formulation adjuvant.

20. A herbicidal composition according to claim 19, further comprising at least one additional pesticide.

21. A herbicidal composition according to claim 20, wherein the additional pesticide is a herbicide or herbicide safener.

22. A method of controlling unwanted plant growth, comprising applying a compound of formula (I) as defined in claim 1 to the unwanted plants or to the locus thereof.

23. (canceled)

24. The compound of claim 1, wherein:
R¹ is hydrogen;
R² is methyl, difluoroethyl, trifluoroethyl, or propargyl;
X is hydrogen, halogen, or C₁-C₃haloalkyl;
Y is hydrogen, C₁-C₃ alkyl, C₁-C₃haloalkyl, or halogen;
Z is independently selected from halogen, methyl, methoxy, and trifluoromethoxy;
n is 0, 1, or 2; and
G is hydrogen.
